(19) United States
(12) Patent Application Publication
(10) Pub. No.: US 2002/0103155 A1
(43) Pub. Date: Aug. 1, 2002

(54) Vectors for the expression of FGF-5 in human cells and uses thereof

(75) Inventor: Denis Gospodarowicz, Lafayette, CA (US)

Correspondence Address: Chiron Corporation, Intellectual Property Department, P.O. Box 8097, Emeryville, CA, 94662-8097, US

(73) Assignee: Chiron Corporation

(21) Appl. No.: 10/023,592

(22) Filed: Dec. 17, 2001

Related U.S. Application Data
(63) Continuation of application No. 08/602,147, filed on Feb. 15, 1996.

(51) Int. Cl. A61K 48/00

(52) U.S. Cl. 514/44

(57) ABSTRACT

A method is described for introducing an FGF-5 nucleic acid sequence into a mammalian host cell. The FGF-5 nucleic acid sequence lacks the signal sequence so that cells that are transformed with the sequence will not become tumirogenic. It is intended that the FGF-5 sequence is introduced into mammalian cells to promote angiogenesis. Preferably, the FGF-5 sequence is introduced into a human patient to treat myocardial ischemia or peripheral vascular disease.

### CROSS REFERENCE TO RELATED APPLICATION

This application is a continuation of U.S. patent application Ser. No. 08/602,147, filed Feb. 15, 1996, the content of which is herein incorporated by reference in its entirety.

### FIELD OF THE INVENTION

The present invention is in the field of gene therapy. More specifically, the present invention is in the field of gene therapy using the FGF-5 gene.

### BACKGROUND OF THE INVENTION

Fibroblast growth factors (FGFs) comprise a family of proteins with related amino acid structure. They are encoded by distinct genes and share sequence homology. Even though there are more than five FGFs, FGFs 1-5 will be discussed here. For example, FGF-1 is acidic FGF, FGF-2 is basic FGF, FGF-3 is int-2, FGF-4 is KFGF or HST, and FGF-5 is described herein.

The FGF-5 of the present invention was originally isolated as an oncogene. See Goldfarb et al. U.S. Pat. Nos. 5,155,217 and 5,238,916, Zhan et al. (1987) “Human Oncogene Detected by a Defined Medium Culture Assay,” Oncogene 1:369-376, Zhan et al. (1988) “The Human FGF-5 Oncogene Encodes a Novel Protein Related to Fibroblastic Growth Factors,” Molecular and Cellular Biology 8:3487-3495, and Bates et al. (1991) “Biosynthesis of Human Fibroblast Growth Factor 5,”Molecular and Cellular Biology 11:1840-1845. The disclosure of each of these patents and articles is hereby incorporated by reference in their entireties. The FGF-5 oncogene nucleic acid sequence was reported in both Goldfarb patents and in the Zhan et al. article (1988, 8). As discussed in each of these references, the FGF-5 gene is an oncogene which can transform cells into a tumorigenic state. Additionally, reports in the literature show that the related genes int-2 and HST can transform cells to be tumorigenic. See Theillet et al. (1985) Oncogene 4:915-922, and Goldfarb et al. (1991) Oncogene 6:65-71.

Consequently, it is the aim of the present invention to use the FGF gene in gene therapy with human patients, while removing the oncogenic potential of this gene.

### SUMMARY OF THE INVENTION

The present invention relates to a method for expressing FGF-5 in vivo, comprising introducing a nucleic acid sequence encoding FGF-5, without a signal sequence, into a vector that can infect mammalian cells and cause these cells to express FGF-5 without causing the cells to become tumorigenic.

The present inventor has discovered that to use FGF-5 in a gene therapy model in human patients, one must remove the signal sequence before administering the gene. Otherwise, the gene therapy may transform normal human cells into tumorigenic cells, which is obviously undesirable.

More specifically, the present invention relates to a gene therapy method for introducing an FGF-5 gene into a human cell of a patient suffering from myocardial ischemia or peripheral vascular disease comprising:

constructing a retroviral vector having a nucleic acid sequence encoding FGF-5, without a signal sequence, having an N terminus of GGGAGAAGCG TCTCGCCCCC AAAG (SEQ ID NO:1), TTCTTCAGCC ACCTGATCCT CAGC (SEQ ID NO:2), ATCCTCAGCG CCTGGGCTCA CGGG (SEQ ID NO:3), CGTCTCGCCC CCAAAGGGCA ACCC (SEQ ID NO:4), or GGGCAACCCG GACCCGCTGC CACT (SEQ ID NO:5) in operable linkage with the appropriate regulatory elements necessary to express the FGF-5 nucleic acid sequence in a human cell, to form the FGF-5 protein; and

introducing the vector into a cellular area in the human patient which is in need of treatment with the FGF-5 protein.

### BRIEF DESCRIPTION OF THE DRAWINGS

FIG. 1 discloses the nucleic acid sequence (SEQ ID NO:6) for the FGF-5 gene which includes the signal sequence.

FIG. 2 is the amino acid sequence (SEQ ID NO:7) for the FGF-5 gene which includes the signal sequence.

FIG. 3 is the nucleic acid sequence (SEQ ID NO:8) for the FGF-5 gene starting at the 22nd amino acid of the sequence of FIG. 1.

FIG. 4 is the amino acid sequence (SEQ ID NO:9) for the FGF-5 gene starting at the 22nd amino acid of the sequence of FIG. 2.

### DETAILED DESCRIPTION OF THE INVENTION

As shown in Goldfarb et al. (U.S. Pat. No. 5,155,217, the disclosure of which is hereby incorporated by reference in its entirety), FGFs 1-5 share a sequence homology between 41 and 50%. For example, column 9 of Goldfarb ('217) shows that there is 45% sequence identity between FGF-5 and basic FGF, 41% sequence homology between FGF-5 and acidic FGF, 52% sequence homology between FGF-5 and KFGF (also called HST), and 50% sequence homology between FGF-5 and int-2 (Goldfarb has used the designation FGF-3 throughout '217 but later changed the identity of their protein to FGF-5). See also Goldfarb et al. U.S. Pat. No. 5,238,916. Basic FGF is more fully disclosed in U.S. Pat. Nos. 5,155,214; 4,994,559; 5,401,701; and 5,439,818. Acidic FGF is disclosed in U.S. Pat. No. 5,312,911. The disclosures of all of the U.S. patents listed above are hereby incorporated by reference in their entireties.

The FGF-5 protein has been shown to be synthesized in vitro in animal cells to yield a 29,500-dalton protein which was a secreted from tumor cells as a glycoprotein containing heterogeneous amounts of sialic acid. Glycosidase treatment suggested that FGF-5 has both N-linked and O-linked sugars. See Bates et al. (1991) “Biosynthesis of Human Fibroblast Growth Factor 5,”Molecular and Cellular Biology 11: 1840-1845, hereby incorporated by reference in its entirety.

The present invention describes the use of the FGF-5 nucleic acid sequence in a gene therapy method whereby the FGF-5 sequence is converted from an oncogene to a protooncogene (non-tumorigenic) before it is introduced into human cells. As described above, the gene sequences are disclosed in the two Goldfarb patents ('217 and '916) and Zhan et al. (1988) “The Human FGF-5 Oncogene Encodes a Novel Protein Related to Fibroblastic Growth Factors,” Molecular and Cellular Biology 8:3487-3495, which are all hereby incorporated by reference in their entireties.

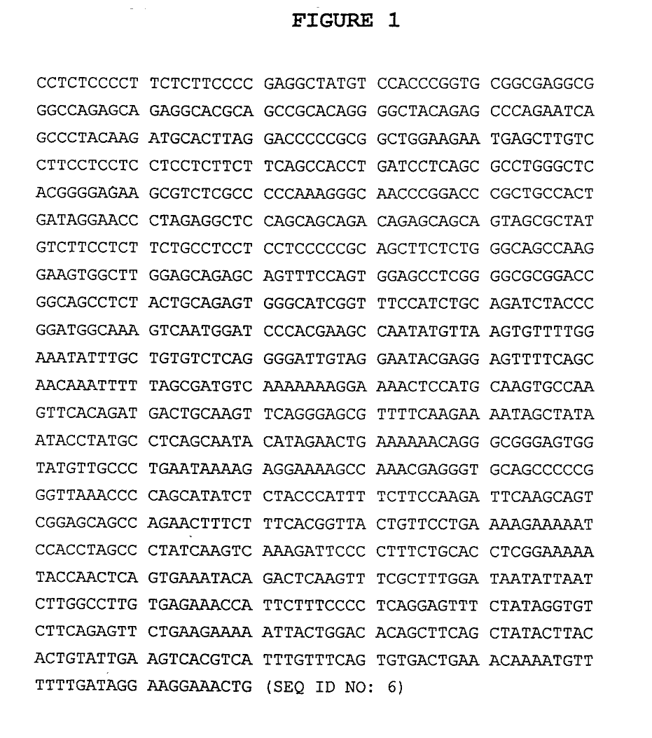

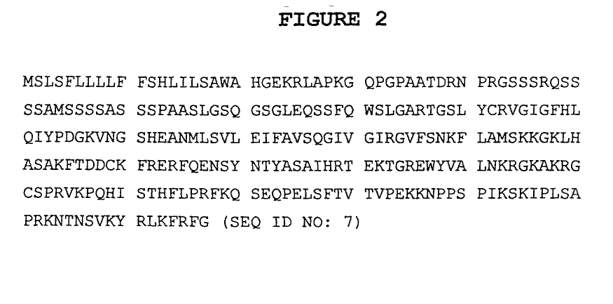

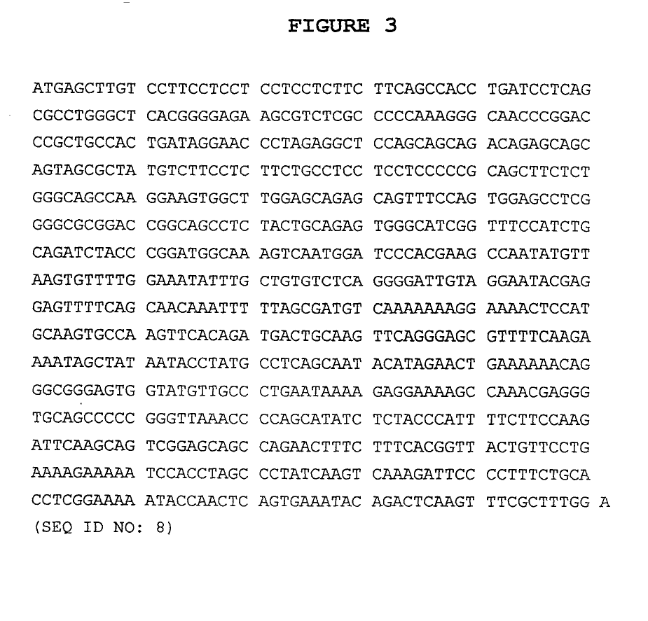

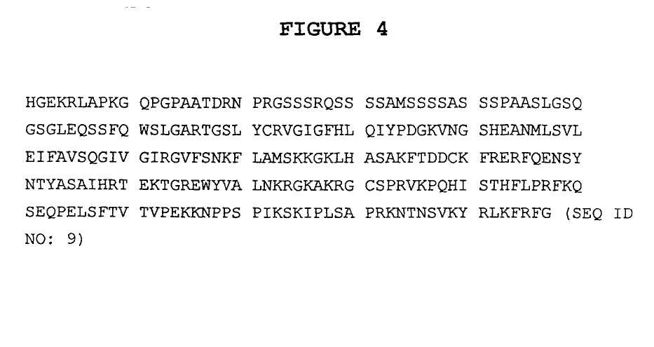

The FGF-5 oncogene protein is a 267 amino acid protein (SEQ ID NO:7) as compared to int-2, which is 240, HSTKS3, which is 206, and acidic and basic FGFs which are both 155 amino acids long. See FIGS. 1 and 2 for the nucleic acid sequence (SEQ ID NO:6) and amino acid sequence (SEQ ID NO:7) of FGF-5, respectively, including the signal sequence. As stated above, the signal sequence of the FGF-5 oncogene must be removed before incorporating it into a gene therapy vector for human use. It is acceptable if enough of the signal sequence is removed so that the tumorigenic properties are eliminated from the FGF-5 molecule described in the Goldfarb patent. Preferably, between 10 and 30 amino acids are removed from the N terminus. More preferably, between 15 and 25 amino acids are removed from the N terminus. Most preferably, the first 22 amino acids are removed from the N terminus. (See Elements et al. (1993) Oncogene 8:1311-1316 which is hereby incorporated by reference in its entirety). See FIGS. 3 and 4 for the FGF-5 nucleic acid sequence (SEQ ID NO:8) and amino acid sequence (SEQ ID NO:9) which begin at the 22nd amino acid of the sequences shown in FIGS. 1 and 2. Elements et al. disclose prokaryotic expression of the mature form of FGF-5 and describe silent mutations in the 5′ end of the cDNA insert that increase the expression levels of FGF-5. The FGF-5 molecule of the present invention preferably contains those mutations.

The present gene therapy method of delivering FGF-5 to local areas in human patients is useful to treat human diseases of the vascular system, as well as enhancing the ability of neural cells to proliferate and for bone growth. See Morrison et al. (1986) “Basic Fibroblast Growth Factor supports the survival of cerebral cortical neurons and primary culture,” Proc. Natl. Acad. Sci. (USA) 83:7537-7541, which are hereby incorporated by reference in their entireties. There is also evidence that FGF-5 is a major muscle-derived survival factor for cultured spinal motor neurons (Hughes et al. (1993) Neuron 10:369-377), that FGF-5 is present in adult mouse central nervous system (Haub et al. (1990) Proc. Natl. Acad. Sci. (USA) 87:8022-8026), that FGF-5 is a regulator of the hair growth cycle (Hebert et al. (1984) Cell 78:1017-1025), that FGF-5 promotes differentiation of cultured rat neurons (Lindholm et al. (1994) European Journal of Neuroscience 6:244-252), that FGF-5 may play a role in limbic system function or dysfunction (Gomez-Pinilla et al. (1993) Brain Research 606:79-86), that FGF-5 can play a role in the biology of the outer retina (Bost et al. (1992) Exp. I. Res. 55:727-34), that basic FGF can ameliorate learning deficits in basal forebrain-lesioned mice (Ishihara et al. (1992) Jpn. J. Pharmacol. 59:7-13). Fibroblasts that have been engineered to express bFGF without a signal sequence have a more robust effect on the viability and function of grafted dopaminergic neurons than with fibroblasts that express bFGF with a signal sequence (see Takayama et al. (1995) Nature Medicine 1:53-58). bFGF appears to be neuroprotective and neurotrophic (see Cheng and Mattson (1991) Neuron 7:1031-1041; Freese et al. (1992) Brain Research 575:351-355; Finkelstein et al. (1993) Stroke 24 (supp. 1):141-143); angiogenic (Baffour et al. (1992) Jour. Vasc. Surg. 16:181-191); and osteogenic (Kawaguchi et al. (1993) Endocrinology 135:774-781; Nagai et al. (1995) Bone 16:367-373; Nakamura et al. (1995) Endocrinology 136:1276-1284; and Mayahara et al. (1993) Growth Factors 9:73-80). Also, it is contemplated that the FGF-5 gene will be useful for many of the uses shown for other FGFs. Accordingly, delivery of the FGF-5 gene will be useful in a variety of vascular, cardiovascular, neuronal, osteogenic, and other indications to correct or regulate cellular dysfunction. Preferably, the FGF-5 gene is administered for angiogenic uses or to support their growth and/or proliferation or neuronal cells. More preferably, the FGF-5 nucleic acid sequence is administered to promote blood vessel growth in myocardial ischemia.

Definitions

The term “polynucleotide” or “nucleic acid sequence” as used herein refers to a polymer of nucleotides of any length, preferably deoxyribonucleotides, and is used interchangeably herein with the terms “oligonucleotide” and “oligomer.” The term refers only to the primary structure of the molecule. Thus, this term includes double- and single-stranded DNA, as well as antisense polynucleotides. It also includes known types of modifications, for example, the presence of labels which are known in the art, methylation, end “caps,” substitution of one or more of the naturally occurring nucleotides with an analog, intemucleotide modifications such as, for example, replacement with certain types of uncharged linkages (e.g., methyl phosphonates, phosphotriesters, phosphoamidates, carbamates, etc.) or charged linkages (e.g., phosphorothioates, phosphorodithioates, etc.), introduction of pendant moieties, such as, for example, proteins (including nucleases, toxins, antibodies, signal peptides, poly-L-lysine, etc.), intercalators (e.g., acridine, psoralen, etc.), chelators (e.g., metals, radioactive species, boron, oxidative moieties, etc.), alkylators (e.g., alpha anomeric nucleic acids, etc.). The term “gene” is used to describe the coding sequence for the polypeptide of interest, for example, FGF-5.

By “genomic” is meant a collection or library of DNA molecules which correspond to the sequence found in chromosomal DNA as opposed to spliced mRNA. By “cDNA” is meant a DNA sequence that hybridizes to a complementary strand of mRNA. “Regulatory” or “control sequence” refers to polynucleotide sequences which are necessary to effect the expression of coding sequences to which they are ligated. The nature of such control sequences differs depending upon the host organism; in eukaryotes, generally, such control sequences include promoters and transcription termination sequences. The term “control sequences” is intended to include, at a minimum, all components whose presence is necessary for expression, and may also include additional components whose presence is advantageous, for example, leader sequences and fusion partner sequences.

“Operably linked” refers to a juxtaposition wherein the components so described are in a relationship permitting them to function in their intended manner. A control sequence “operably linked” to a coding sequence so that expression of the coding sequence is achieved under conditions compatible with the control sequences.

A “vector” or “plasmid” is a nucleic acid sequence in which another polynucleotide segment is attached, so as to bring about the replication and/or expression of the attached segment in a host cell. Vectors are used routinely in recombinant DNA techniques. Any extrachromosomal small genome such as a plasmid, phage, or virus is a potential vector.

“Retroviral vector” is a vector derived from a retrovirus and it has the capability to insert a gene or DNA fragment into the host chromosomal genome by a recombinational event, so that the DNA fragment can be expressed in a host cell. See Singer and Berg (1991) Genes and Genomes, pp. 310-314 (Mill Valley, Calif.) which is hereby incorporated by reference. Retroviruses are RNA viruses (the viral genome is RNA). The genomic RNA is reverse transcribed into DNA after it enters the cell and then it is integrated stably and efficiently into the chromosomal DNA of transduced cells. See Mulligan (1983) in Experimental Manipulation of Gene Expression, ed. M. Inouye, pp. 155-173; Mann et al. (1983) Cell 33:153-159; Cone and Mulligan (1984) Proc. Natl. Acad. Sci. (USA) 81:6349-6353, which are hereby incorporated by reference in their entireties. “Transformation”, as used herein, refers to the insertion of an exogenous polynucleotide into a host cell, irrespective of the method used for the insertion, for example, direct uptake, particle mediated, transduction, f-mating, or electroporation. The exogenous polynucleotide may be maintained as a non-integrated vector, for example, a plasmid, or alternatively, may be integrated into the host genome. Examples of particle mediated transduction are shown in U.S. Pat. Nos. 4,945,050 and 5,149,655, which are hereby incorporated by reference in their entireties.

“Homology” refers to the degree of similarity between x and y. The correspondence between the sequence from one form to another can be determined by techniques known in the art. For example, they can be determined by a direct comparison of the sequence information of the polynucleotide. Alternatively, homology can be determined by hybridization of the polynucleotides under conditions which form stable duplexes between homologous regions (for example, those which would be used prior to S₁ digestion), followed by digestion with single-stranded specific nuclease(s), followed by size determination of the digested fragments.

As used herein, x is “heterologous” with respect to y if x is not naturally associated with y in the identical manner; i.e., x is not associated with y in nature or x is not associated with y in the same manner as is found in nature.

As used herein, the term “protein” or “polypeptide” refers to a polymer of amino acids and does not refer to a specific length of the product; thus, peptides, oligopeptides, polypeptides, proteins, and polyproteins, as well as fragments of these, are included within this definition. This term also does not refer to, or exclude, post expression modifications of the protein, for example, glycosylations, acetylations, phosphorylations, and the like. Included within the definition are, for example, proteins containing one or more analogs of an amino acid (including, for example, unnatural amino acids, etc.), proteins with substituted linkages, as well as other modifications known in the art, both naturally occurring and non-naturally occurring.

A polypeptide or protein or amino acid sequence “derived from” or “coded by” a designated nucleic acid sequence refers to a polypeptide having an amino acid sequence identical to that of a polypeptide encoded in the sequence, or a portion thereof wherein the portion consists of at least 3-5 amino acids, and more preferably at least 8-10 amino acids, and even more preferably at least 11-15 amino acids, or which is immunologically identifiable with a polypeptide encoded in the sequence. This terminology also includes a polypeptide expressed from a designated nucleic acid sequence.

“Alleles” and “variants” refers to a polypeptide that differs from the native specified protein by virtue of one or more amino acid substitutions, deletions, or insertions. The amino acid substitutions can be conservative amino acid substitutions or substitutions to eliminate non-essential amino acid residues such as to alter a glycosylation site, a phosphorylation site, an acetylation site, or to alter the folding pattern by altering the position of the cysteine residue that is not necessary for function, etc. Conservative amino acid substitutions are those that preserve the general charge, hydrophobicity/hydrophilicity and/or steric bulk of the amino acid substituted, for example, substitutions between the members of the following groups are conservative substitutions: Gly/Ala, Val/Ile/Leu, Asp/Glu, Lys/Arg, Asn/Gln, and Phe/Trp/Tyr.

“Signal sequence” is used to describe the N-terminal amino acids that enable the polypeptide to be transported outside the boundaries of the cells in which it is made. As stated above, it is this sequence that enables the FGF-5 nucleic acid sequence to transform cells into a tumorigenic state. In FGF-5, it is the first 59 or, more preferably, the first 61 amino acids at the N terminus that constitute the signal sequence.

The term “cardiovascular indication” as used herein refers to a diagnosis or presumptive diagnosis of cardiovascular disease or conditions affecting the heart that are associated with atheroscerosis, ischemic syndromes, cardiomyopathies, arrhythmias, dysrrhythmias, hypertension, and infections. The diagnosis can be made based on pain, fatigability, weakness, palpitations, and systemic symptoms that may be due to the cardiac disease or that may accompany it. Determination of a cardiovascular indication may include a physical exam and other non-invasive diagnostic procedures including radionuclide imaging, positon emission tomography, magnetic resonance imaging, echocardiography, and can also include venous and arterial cannulation and pulmonary and cardiac catheterization used in diagnosis of the cardiac condition.

The term “administering to intrapericardially” or “administering into the pericardial space” as used herein refers to any method of administration that effects delivery of a therapeutic agent into the pericardial space. The pericardial space may be the entire region comprising the pericardial space, or only a part of it. The term “administering into pericardial space” is synonymous with the terms “intrapericardial delivery” and “pericardial delivery”, and can include delivery to subregions of the pericardial space that form interfaces between the pericardial space and the tissue that surrounds and forms it. The administration into pericardial space can be accomplished by, for example, the following means of administration including injection, laser, catheter, pump. Intrapericardial delivery of the polynucleotides and the drugs of the invention can be accomplished by the methods of such delivery as disclosed in, for example, U.S. Pat. Nos. 5,137,510, 5,269,326, and 5,213,570, herein incorporated by reference.

Vectors and Expression Systems

The following expression systems describe vectors, promoters, and regulatory elements that are useful for gene therapy applications for the delivery of the FGF-5 polynucleotide. Vectors and expression systems useful for the present invention include viral and non-viral systems. Example viral delivery systems include retroviruses, adenoviruses, adeno-associated viruses (AAV), sindbis and herpes viruses. In one aspect of the present invention, the viral vector is capable of integrating the FGF-5 nucleic acid sequence into the host cell genome for long-term expression. Examples of vectors that can integrate in this fashion are retroviruses and AAV. One preferred retrovirus is a murine leukemia virus. However, it may be preferred to avoid integration into the host cell genome. For example, when short-term administration of FGF-5 is required, long-term expression can be unnecessary and possibly undesirable. Non-viral vectors include naked DNA and DNA formulated with cationic lipids or liposomes.

Preferably, the FGF-5 nucleic acid coding sequence is administered in one of the above systems to a patient's cells without the signal sequence. The description below is directed to means for including the FGF-5 coding sequence in a larger sequence that will facilitate expression of the FGF-5 polypeptide.

Retroviral vectors are produced by genetically manipulating retroviruses. Retroviral vectors are effective for integration into the host cell genome, as explained above. However, they only infect dividing cells. Retroviral vectors contain RNA. In the present invention the viral RNA vector contains the FGF-5 gene, and once it enters the cell, it is reverse transcribed into DNA and stably integrated into the host cell genome.

The wild type retrovirus genome contains three genes: the gag, pol, and env genes, which are flanked by the long terminal repeat (LTR) sequences. The gag gene encodes the nucleocapsid proteins, the pol gene encodes the viral enzymes including reverse transcriptase and integrase, the env gene encodes the viral envelope glycoproteins. The 5′ and 3′ LTRs serve to promote transcription and polyadenylation of virion RNAs. Adjacent to the 5′ LTR are sequences necessary for reverse transcription of the genome (the tRNA primer binding site) and for efficient encapsulation of viral RNA into particles (the Psi site). See Mulligan (1983) in Experimental Manipulation of Gene Expression, ed. M. Inouye, pp. 155-173; Mann et al. (1983) Cell 33:153-159; Cone and Mulligan (1984) Proc. Natl. Acad. Sci. (USA) 81:6349-6353.

More specifically, the present invention contemplates constructing a vector in which the gag, pol, and env genes are removed and replaced with the FGF-5 gene. The LTR, psi sequence, and primer binding sites are also present to facilitate vector replication. The vector is transformed into a packaging animal cell line which contains the gene sequences for the gag, pol, and env genes in its genome and which constitutively express those proteins. These proteins are usually expressed from a heterologous promoter (e.g. CMV) and the genes are not operably linked to sequences (such as psi, LTR which are required for viral replication). This cell will make empty viral particles and is a recipient for the vector described above which contains the FGF-5 gene, the psi and primer binding sequences, as well as the LTR sequences. The cell can be transiently transfected with the vector to produce the product (viral particle with the FGF-5 vector). Preferably, the product virions are used to infect a second packaging cell line which then can permanently produce the viral particles.

The retroviral vector can be packaged by transfecting the FGF-5 nucleic acid sequence into cells expressing the gag-pol and env genes. These “packaging cell lines” are mammalian tissue culture cell lines which express structural proteins of a retrovirus and produce retrovirus-like particles. They are incapable of producing infectious virions. Transfecting retroviral vectors (with the FGF-5 nucleic acid sequence) into packaging cell lines results in the production of retroviral vector particles with the desired genetic construction. Packaging cell lines are publically available and include Crip, GPE86, PA317, and PG13. See Miller et al. (1991) J. Virol. 65:2220-2224, Cone et al. (1988) Proc. Natl. Acad. Sci. (USA) 85:6460-6464, Eglitis et al. (1988) Biotechniques 4:608-614, Miller et al. (1990) Human Gene Ther. 1:5-14, which are all hereby incorporated by reference in their entireties.

Also, AAV are advantageous because they replicate to a high titer, they integrate efficiently, are not pathogenic to humans, are stable, easy to purify, and they infect non-dividing cells. An AAV vector is constructed by inserting the FGF-5 coding sequence, under the control of a suitable promoter/enhancer, between the AAV LTRs, which are the only sequences required in cis for AAV replication. This DNA construct is transfected into a suitable human cell line in the presence of another plasmid which expresses Rep and CAP, the AAV coding regions needed for replication. At a suitable time post-transfection, the cells are infected with a helper virus, such as Adenovirus or Herpes Simplex virus. After infection, vector particles carrying the FGF-5 gene are harvested from these cells. The AAV particles are purified from contaminating Adenovirus or Herpes Virus by standard protocols.

Adenovirus is advantageous because it infects a wide variety of cells, infects non-dividing cells, produces a high titer, the biology is well understood, and it can accept large inserts. The adenovirus gene expression is controlled by a cascade of genes. For example, the gene expression order is “immediate early”, “early”, DNA synthesis, and late or structural genes. These genes are turned on in sequence. The master gene that is turned on first is E1A. One preferred embodiment would involve replacing the E1A gene with the FGF-5 gene and transfecting this vector into cells that constitutively produce El A, such as 293 cells which are publically available. The vector contains all the genes necessary for virion production and the cell line provides the missing E1A protein. Consequently, the virion is produced which contains the FGF-5 sequence.

One non-viral system that can be used is the T7/T7 system. Here a short promoter sequence recognized by the bacterial virus T7 polymerase is placed on a vector upstream of the FGF-5 gene. The vector can then be inserted into cells and the missing T7 polymerase can be added to obtain gene transcription. Alternatively, a vector containing the following sequences can be made, the T7 promoter sequence, the T7 polymerase gene, another copy of the T7 promoter sequence, and the FGF-5 gene. In this embodiment, the vector is transformed into cells and simply requires a small amount of T7 polymerase to initiate. Thereafter, the vector directs the manufacture of its own polymerase.

Although the methodology described is believed to contain sufficient details to enable one skilled in the art to practice the present invention, other items not specifically exemplified, such as plasmids, can be constructed and purified using standard recombinant DNA techniques described in, for example, Sambrook et al. (1989) Molecular Cloning: A Laboratory Manual (2d edition, Cold Spring Harbor Press, Cold Spring Harbor, N.Y.), and Ausubel et al. (1994) Current Protocols in Molecular Biology (Greene Publishing Associates and John Wiley & Sons, New York, N.Y.) under the current regulations described in United States Dept. of HEW, NATIONAL INSTITUTE OF HEALTH (NIH) GUIDELINES FOR RECOMBINANT DNA RESEARCH. These references include procedures for the following standard methods: cloning procedures with plasmids, transformation of host cells, cell culture, plasmid DNA purification, phenol extraction of DNA, ethanol precipitation of DNA, agarose gel electrophoresis, purification of DNA fragments from agarose gels, and restriction endonuclease and other DNA-modifying enzyme reactions.

Gene therapy can be practiced according to the invention by genes that are under regulatory control of appropriate regulatory sequences for transformation or infection of myocytes, cells within the pericardium, cells at the epicardium, or any cells in a region of the heart accessible to an intrapericardially delivered gene. When the genes are directed to nerve cells, the genes must be under the appropriate regulatory elements that enable expression in those cells. Gene therapy can be practiced as follows using coding regions for any therapeutic appropriate for treatment of a cardiovascular or neural indication.

As explained above, gene therapy strategies for delivery of the FGF-5 gene nucleic sequence can utilize viral or non-viral vector approaches in in vivo or ex vivo modality. Expression of such coding sequence can be induced using endogenous mammalian, viral, or other heterologous promoters. Expression of the coding sequence in vivo can be either constitutive or regulated.

For delivery using viral vectors, any of a number of conventional viral vectors can be used, as described in Jolly (1994) Cancer Gene Therapy 1:51-64. Promoters that are suitable for use with these vectors are also conventional in the art and include the Moloney retroviral LTR, CMV promoter, and the mouse albumin promoter. Virus competent for one round of replication can be produced and injected directly into the animal or humans or by transduction of an autologous cell ex vivo, followed by injection in vivo as described in Zatloukal et al. (1994) Proc. Natl. Acad. Sci. USA 91:5148-5152.

Delivery

Preferably, the FGF-5 gene is administered to the local area of the pericardium or neural cells. More preferably, the FGF-5 gene is delivered to the pericardium without the signal sequence. The FGF-5 nucleic acid sequence may be delivered into the pericardial space by any method conventional in the art, such as that described in Barr et al. 1994) Gene Therapy 1:51-58. Barr et al. describe gene delivery via catheter-mediated infusion of replication defective adenovirus into the coronary arterial circulation. High level expression of an exogenous gene was obtained throughout the thickness of the ventricular and arterial walls within the distribution of the injected coronary artery. The FGF-5 nucleic acid sequence may be linked to tissue-specific promoters or leader sequences for expression in cardiac muscle cells, for example, the untranslated leader sequence of dystrophin DNA, or regulatory regions in the muscle creatine kinase gene such as that described in Cox et al. (1993) Nature 364:725-729.

Delivery of genes to the intrapericardial space is a safer and more effective method of accomplishing myocardial gene therapy. Accordingly, delivery of genes to the pericardial space does not require mechanical violation of the myocardium as does direct myocardial injection. Because intrapericardially delivered agents have access to the entire myocardial surface the ease and effectiveness with which genes can be delivered to large areas of myocardium is increased. Access to the coronary circulation causes perfusion of the entire heart with these agents. Also, the pericardium is more easily transducible than the myocardium and, thus, that expression of gene products in the pericardial space retains access to myocardium.

Furthermore, the exposure time of nucleic acids and/or viruses to cells, which is an important determinant of transduction or infection efficiency, increases. Genetic agents deposited in the pericardial space are not subject to rapid dilution, drainage, or dissipation due to blood flow or lymphatic clearance, and thus have much longer exposure times than vascularly delivered agents, also increasing the transduction or infection efficiency of the genes. Such an advantage achieved by the method of the invention, translates into much higher transduction or infection efficiency with genes and/or viruses in either the myocardium or the pericardium than is achievable in the coronary vessel. Lastly, because pericardium is highly efficient at expressing certain proteins and in some cases is even more efficient than myocardium at this task, the method of the invention is a new and improved method of delivery of genes for gene therapy for treatment of a cardiovascular indication.

Practice of the invention also includes, for example, delivering the FGF-5 genes into the pericardial space, optionally in combination with cardiovascular therapeutics, in liposomal compositions, including heterovesicular liposomes. Delivery in liposomes increases the efficacy of the gene or cardiovascular therapeutics, reduces the dosage requirements, and augments the benefits of any cardiovascular therapeutic delivered into the pericardial space.

Additionally, the FGF-5 gene can be delivered to nerve tissue. Actual delivery methods may vary, depending on the sites of the nerves to be affected. For example, administration to nerve tissue may be by encapsulating the FGF nucleotide sequence in a herpes virus which will specifically target nerve cells.

For in vivo therapy, the coding sequence can be delivered into the intrapericardial space by direct injection, or into pericardial tissue by delivery such as, for example, those systems described in U.S. Pat. Nos. 5,137,510, 5,213,570, and 5,269,326. Promoters suitable for use in this manner include endogenous and heterologous promoters such as those described herein. Any promoter appropriate for the expression of the gene selected for the therapy is contemplated by the method of the invention. The coding sequence can be injected in a formulation comprising a buffer that can stabilize the coding sequence and facilitate transduction thereof into cells and/or provide targeting, as described in Zhu et al. (1993) Science 261:209-211.

Expression of the FGF-5 coding sequence in vivo (by either viral or non-viral vectors) can be regulated by use of regulated gene expression promoters as described in Gossen et al. (1992) Proc. Natl. Acad. Sci. (USA) 89:5547-5551. For example, the coding sequence selected for the therapy can be regulated by tetracycline responsive promoters. These promoters can be regulated in a positive or negative fashion by treatment with the regulator molecule. Additionally, the FGF-5 gene may be introduced into cells under the control of promoters which are activated using radiotherapy. For example, U.S. Pat. No. 5,205,152 entitled “Cloning and Expression of Early Growth Regulatory Protein Genes” shows that the Egr-1 gene is one of the best radiation induced genes and may be activated by exposure to radiation. WO 92/11033 disclosed genetic constructs which comprise an enhancer-promoter region which is responsive to radiation, and at least one structural gene whose expression is controlled by the enhancer-promoter. The U.S. Patent and the PCT application are hereby incorporated by reference in their entireties.

For non-viral delivery, the FGF-5 coding sequence can be inserted into conventional vectors that contain conventional control sequences for high level expression, and then incubated with synthetic gene transfer molecules such as polymeric DNA-binding cations like polylysine, protamine, and albumin, linked to cell targeting ligands such as asialoorosomucoid, as described in Wu and Wu (1987) J. Biol. Chem. 262:4429-4432; insulin, as described in Hucked et al. Biochem. (1990) Pharmacol. 40:253-263; galactose, as described in Plank et al. (1992) Bioconjugate Chem. 3:533-539; lactose, as described in Midoux et al. (1993) Nucleic Acids Res. 21:871-878; or transferrin, as described in Wagner et al. (1990) Proc. Natl. Acad. Sci. (USA) 87:3410-3414. Other delivery systems include the use of liposomes to encapsulate DNA comprising the gene under the control of a variety of tissue-specific or ubiquitously active promoters, as described in Nabel et al. (1993) Proc. Natl. Acad. Sci. (USA) 90:11307-11311, and Philip et al. (1994) Mol. Cell Biol. 14:2411-2418. Further non-viral delivery suitable for use includes mechanical delivery systems such as the biolistic approach, as described in Woffendin et al. (1994) Proc. Natl. Acad. Sci. (USA) 91(24): 11581 -11585. Moreover the coding sequence and the product of expression of such can be delivered through deposition into the pericardial space of photopolymerized hydrogel materials such as Focalgel®. Furthermore, the FGF-5 gene sequence can be inserted into a host cell by direct uptake or particle-mediated transduction. The FGF-5 sequence may be maintained as a non-integrated vector, for example, a plasmid, or alternatively, may be integrated into the host genome. Examples of particle-mediated transduction are shown in U.S. Pat. Nos. 4,945,050 and 5,149,655, which are hereby incorporated by reference in their entireties.

As stated above, naked DNA can be administered to muscle tissue. See Wolff, J. A. et al. (1990), entitled “Direct Gene Transfer into Mouse Muscle In Vivo,” Science 247:1465-1468; Kitsis et al. (1991) “Hormonal Modulation of a Gene Injected into Rat Heart In Vivo,” Proc. Natl. Acad. Sci. 88:4138-4142; Lin et al. (1990) “Expression of Recombinant Genes in Myocardium In Vivo after Direct Injection of DNA,” Circulation 82:2217-2221; and Buttrick et al. (1992) “Behavior of Genes Directly Injected into Rat Heart In Vivo,” Circ. Res. 70:193-198. The above references are hereby incorporated by reference in their entireties.

To practice one aspect of the invention, the diagnosis of a cardiovascular condition is made, and the appropriate dosages are determined on the basis of the diagnosis. The invention is practiced to prevent, reduce or treat a cardiovascular condition.

The method of the invention applies to any cardiovascular indication, for example a diagnosis of: (1) atherosclerosis, and conditions that predispose one to pathological atherosclerotic plaque development in the coronary arteries including lipid/cholesterol deposition, macrophage/inflamrnatory cell recruitment, plaque rupture, thrombosis, platelet deposition, neointimal proliferation; (2) ischemic syndromes and attendant syndromes, including but not limited to myocardial infarction, stable and unstable angina, coronary artery restenosis following percutaneous transluminal, coronary angioplasty, reperfusion injury; (3) cardiomyopathies, including but not limited to cardiomyopathies caused by ischemic syndromes, cardiotoxins such as alcohol and chemotherapeutic agents like adriamycin, infections, such as viral, cytomegalovirus (CMV), and parasitic (trypanosoma cruzi), hypertension, metabolic diseases (including but not limited to uremia, beriberi, glycogen storage disease), radiation, neuromuscular disease (such as Duchenne's muscular dystrophy), infiltrative diseases (including but not limited to sarcoidosis, hemochromatosis, amyloidosis, Fabry's disease, Hurler's syndrome), trauma, and idiopathic causes; (4) a/dysrrhythmias (including but not limited to a/dysrrhythmias resulting from the same causes listed above for cardiomyopathies); (5) infections (including bacterial, viral, fungal, and parasitic causes); (6) cardiac tumors; (7) inflammatory conditions (including but not limited to myocarditis, pericarditis, endocarditis, immune cardiac rejection and conditions resulting from idiopathic, autoimmune, or connective tissue diseases); and (8) hypertension.

The FGF-5 nucleotide sequence can be administered to the pericardial space and expressed in the heart tissue, including but not limited to, for example, pericardial tissue, myocardial tissue, epicardial tissue, or perivascular tissue. The sequence can be placed in a vector, such as a viral vector, or a plasmid vector. The polynucleotides may be presented into the pericardial space in any formulation commonly known in the art including buffers, excipients, gels, matrices and polymers. Appropriate formulations for the polynucleotides administered intrapericardially in the practice of the invention also include liposomal preparations such as, for example, those disclosed in U.S. Pat. No. 5,422,120, WO 95/13796, WO 94/23697, WO 91/14445 and EP 524,968 B1, particularly including the heterovesicular liposomal preparations disclosed in these patents and applications.

The present invention will now be illustrated by reference to the following examples which set forth particularly advantageous embodiments. However, it should be noted that these embodiments are illustrative and are not to be construed as restricting the invention in any way.

### EXAMPLE 1

The coding sequence for FGF-5, without the signal sequence, is isolated by standard recombinant DNA techniques and placed in a retroviral vector and encapsulated in viral envelope for delivery intrapericardially. The retrovirus is delivered by laparoscopic cannulation or direct injection into the pericardial space of a patient who is suffering from myocardial ischemia or peripheral vascular disease. Alternatively, the coding sequence is placed in a plasmid vector and the vector is likewise delivered into the pericardial space. The coding sequences are linked with appropriate regulatory sequences and are delivered into the pericardial space by laparoscopic cannulation or direct injection. The FGF-5 nucleic acid sequence is expressed by the patient's cells in the local area of the release and the FGF-5 protein induces the formation of new blood vessels.

The present invention has been described with reference to specific embodiments. However, this application is intended to cover those changes and substitutions which may be made by those skilled in the art without departing from the spirit and the scope of the appended claims.

All publications and patent applications mentioned in the specification are indicative of the level of those skilled in the art to which this invention pertains. All publications and patent applications are herein incorporated by reference to the same extent as if each individual publication or patent application was specifically and individually indicated to be incorporated by reference.

That which is claimed:

1. A method for expressing human FGF-5 in human cells without inducing tumorigenicity, comprising:
a) introducing a nucleic acid sequence encoding human FGF-5 without a signal sequence (hereinafter “FGF-5 fragment”) into a replication defective viral vector that infects human cells, said viral vector being an adenoviral vector or an AAV vector, said FGF-5 having angiogenic activity but not tumorigenic activity, said nucleic acid sequence being operably linked to a promoter for expression in said human cells; and
b) infecting said human cells with said vector containing said sequence to cause said human cells to express an angiogenic inducing amount of said FGF-5 without causing said human cells to become tumorigenic.

2. The method of claim 1, wherein the nucleic acid sequence has the sequence TTCTTCAGCC ACCTGATCCT CAGC (SEQ ID NO:2) encoding the N terminus of said FGF-5 fragment.

3. The method of claim 1, wherein the nucleic acid sequence has the sequence ATCCTCAGCG CCTGGGCTCA CGGG (SEQ ID NO:3) encoding the N terminus of said FGF-5 fragment.

4. The method of claim 1, wherein the nucleic acid sequence has the sequence GGGAGAAGCG TCTCGCCCCC AAAG (SEQ ID NO:1) encoding the N terminus of said FGF-5 fragment.

5. The method of claim 1, wherein the nucleic acid sequence has the sequence CGTCTCGCCC CCAAAGGGCA ACCC (SEQ ID NO:4) encoding the N terminus of said FGF-5 fragment.

6. The method of claim 1, wherein the nucleic acid sequence has the sequence GGGCAACCCG GACCCGCTGC CACT (SEQ ID NO:5) encoding the N terminus of said FGF-5 fragment.

7. The method of claim 1, wherein said FGF-5 fragment comprises the FGF-5 of SEQ ID NO:7 lacking the first 59 of the first 61 residues from its N terminus.

8. The method of claim 1, wherein the replication defective viral vector is an adenoviral vector.

9. The method of claim 1, wherein the replication defective viral vector is administered into the intrapericardial space of a human patient in need of angiogenesis.

10. The method of claim 9, wherein the replication defective viral vector is used to treat myocardial ischemia or peripheral vascular disease.

11. A method for introducing a non-tumorigenic FGF-5 gene into a human cell in an area of the human myocardium afflicted by myocardial ischemia comprising:
a) constructing a viral vector that infects human cells, said viral vector being an adenoviral vector or an AAV vector having a nucleic acid sequence encoding human FGF-5 without a signal sequence (hereinafter “FGF-5 fragment”) in operable linkage with the appropriate regulatory elements necessary to express the nucleic acid sequence in a human cell, said nucleic acid sequence having the sequence GGGAGAAGCG TCTCGCCCCC AAAG (SEQ ID NO:1) encoding the N terminus of the FGF-5 fragment; and
b) injecting said viral vector in an area of said myocardium afflicted by myocardial ischemia to infect said cells in said area with said vector, said FGF-5 fragment being expressed in said cells in an angiogenic inducing amount without inducing said cells to become tumorigenic.

12. The method of claim 11, wherein said FGF-5 fragment comprises the FGF-5 of SEQ ID NO:7 lacking the first 59 of the first 61 residues from its N terminus.

13. A method for introducing a non-tumorigenic human FGF-5 gene into a human heart cell in vivo comprising:
a) constructing a viral vector that infects human cells, said viral vector being an adenoviral vector or an AAV vector having a nucleic acid sequence encoding human FGF-5 without a signal sequence (hereinafter “FGF-5 fragment”) in operable linkage with the appropriate regulatory elements necessary to express the nucleic acid sequence in a human cell; and
b) injecting said viral vector into the pericardial space of a human patient, said vector infecting human heart cells enclosed within said pericardial space and expressing said FGF-5 fragment therein, said FGF-5 fragment being free of tumorigenic activity.

14. The method of claim 13, wherein said FGF-5 fragment comprises the FGF-5 of SEQ ID NO:7 lacking the first 59 of the first 61 residues from its N terminus.

15. A pharmaceutical composition comprising:
a) an angiogenically inducing effective amount of a viral expression vector that infects a human cell, said viral expression vector being an adenoviral vector or an AAV vector and comprising a nucleic acid sequence encoding human FGF-5 without a signal sequence (hereinafter “FGF-5 fragment”), said nucleic acid sequence being operably linked to a promoter for expression in said human cell, said FGF-5 fragment having angiogenic activity but not tumorigenic activity; and
b) a pharmaceutically acceptable carrier.

16. The pharmaceutical composition of claim 15, wherein said viral vector is an adenoviral vector.

17. The pharmaceutical composition of claim 15, wherein said viral vector is replication defective.

18. A pharmaceutical composition comprising:
a) a viral expression vector that infects a human cell, said viral expression vector being an adenoviral vector or an AAV vector and comprising a nucleic acid sequence encoding human FGF-5 without a signal sequence (hereinafter “FGF-5-Fragment”), said nucleic acid sequence being operably linked to a promoter for expression in said human cell, said FGF-5 fragment having angiogenic activity but not tumorigenic activity; and
b) a pharmaceutically acceptable carrier.
